(19) United States
(12) Patent Application Publication
(10) Pub. No.: US 2018/0333237 A1
(43) Pub. Date: Nov. 22, 2018

(54) APPARATUS AND METHOD FOR ANATOMIC RESTORATION DENTAL IMPLANT HAVING A TWO-PIECE REINFORCED ZIRCONIUM DIOXIDE BASE AND TITANIUM INSERT

(71) Applicants: Fereidoun Daftary, Beverly Hills, CA (US); Oded Bahat, Pacific Palisades, CA (US)

(72) Inventors: Fereidoun Daftary, Beverly Hills, CA (US); Oded Bahat, Pacific Palisades, CA (US)

(21) Appl. No.: 15/668,214

(22) Filed: Aug. 3, 2017

Related U.S. Application Data
(60) Provisional application No. 62/488,736, filed on Apr. 22, 2017.

(51) Int. Cl. A61C 13/10; A61C 5/77; A61B 17/86

(52) U.S. Cl. CPC A61C 13/1003; C04B 35/119; A61B 17/863; A61C 5/77

(57) ABSTRACT

A two-piece anatomic dental implant with a Zirconium Dioxide base and a titanium insert. The Zirconium Dioxide base having a milled shaft into which a titanium insert is press fit retained with members to prevent rotation of the titanium insert within the Zirconium Dioxide implant. The present invention only allows metal screws from attachments to be threaded into a metal titanium insert and not threaded into a Zirconia Implant base. The invention includes an improved design for a healing cap providing minimal contact between the stronger material such as titanium and the Zirconium Dioxide.

### CROSS-REFERENCE TO RELATED APPLICATION

This patent application claims priority to Provisional Application Ser. No. 62/488,736 filed on Apr. 22, 2017, now pending.

### BACKGROUND OF THE INVENTION

### 1. Field of the Invention

The present invention relates to the field of cosmetic and restoration dentistry utilized by dentists if at least one tooth is no longer in a patient's mouth. Specifically, this invention relates to the field of implants used in cosmetic and tooth restoration dentistry.

### 2. Description of the Prior Art

The following 9 patents and published patent applications are the closest prior known to the present inventors at the time of filing this patent application.

1. U.S. Pat. No. 6,280,193 issued to Guy Peatier on Aug. 28, 2001 for “Zirconium Oxide Dental Implant With Internal Thread” (hereafter the “Peatier patent”);
2. WIPO (World intellectual property organization) Patent Publication WO2006/035011 A1 published on Sep. 26, 2005 for “Ceramic Enossal Tooth Implant” (hereafter the “WIPO patent”);
3. United States Published Patent Application No. 2006/0246399 by Peter A. Ehrl, and published on Nov. 2, 2006 for “Two-Part Ceramic Dental Implant”;
4. U.S. Pat. No. 8,740,615 issued to Teruo Ishiwata on Jun. 3, 2014 for “Implant, Implant Body, And Abutment Body”;
5. United States Published Patent Application No. 2015/0037759 by Zipprich et al., published on Feb. 5, 2015 for “Dental Implant System And Method For Producing A Dental Implant”;
6. U.S. Pat. No. 8,961,178 issued to Junichi Hayashi on Feb. 24, 2015 for “Method For Manufacturing Dental Implant And Dental Implant”;
7. U.S. Pat. No. 8,986,007 issued to Chun-Leon Chen on Mar. 24, 2015 for “Restorable Zirconium Dioxide Based One Piece Dental Implant”;
8. European Patent No. EP2113221B1 and granted publication on Oct. 7, 2015 for “Dental Implant Made of Ceramics”; and
9. U.S. Pat. No. 9,452,111 issued to Klein Woolthuis on Sep. 27, 2016 for “Dental Implant System And Associated Insertion Instruments”.

The Peatier patent attempted to create an implant with an abutment and crown without providing time for the gum tissue to heal and does not provide sufficient time for the bone to grow into the implant before affixing a crown.

The WIPO patent has a major defect in that a metal screw is threaded into Zirconia.

For the remaining prior art, numerous attempts, as shown in the prior art, have been created to combine the aesthetically pleasing benefits of Zirconium Dioxide with the strength benefits of a strong metal such as titanium for a two-piece implant system. The prior art fails to address the issues of how the metal strengthening portion of the implant should connect with the Zirconium Dioxide portion to prevent fracturing of the Zirconium Dioxide.

Specifically, it is found throughout the prior art that male metal screws are threaded into bases made of Zirconium Dioxide. This method of a two-piece system results in similar fractures because the metal which comes in direct contact with the Zirconium Dioxide can cause the Zirconium Dioxide portion to fracture. As a result, there is a significant need in this highly evolving area of implant dentistry to provide a two-piece system that improves the apparatus when the titanium screw from an attachment comes in contact with the aesthetically pleasing Zirconium Dioxide.

The present inventors are innovators in the field of implant dentistry. Reference is made to several figures from a prior art patent discussing an earlier innovation by one of the same two inventors of the present invention, namely U.S. Pat. No. 5,759,034 issued to Fereidoun Daftary on Jun. 2, 1998 for “ANATOMICAL RESTORATION DENTAL IMPLANT SYSTEM FOR POSTERIOR AND ANTERIOR TEETH” (hereafter the “'034 patent”). The '034 patent is discussed herein to provide a background to better understand the significant improvement disclosed and claimed in the present patent application. Specifically, the following discussion and figures are taken from the '034 patent.

Referring to FIGS. 1, 2, 3, and 5 of the '034 application, the anatomic dental implant fixture 16 consists of a head section 26 with a widened proximal end 28 and an elongated tapered shaft section 30 with a narrow distal end 32. The head section 26 is integrally connected to the tapered shaft section 30. and thereby forms a one-piece implant fixture 16. This prevents bacteria or other infection growth between the head section and the shaft section of the implant fixture. The implant is made of metal.

The '034 patent discloses a one-piece implant fixture. This implant was originally made of metal. One improvement in the present invention implant fixture is that it is made out of Zirconium Dioxide. The benefit of Zirconium Dioxide was that it resembled tooth color, as opposed to metal. One problem identified by the present inventors is that an implant made of Zirconium Dioxide can result in a shattered implant fixture when a tightening screw is threaded into the implant to affix a healing cap. Therefore, there is a significant need to create an implant fixture that is both aesthetically pleasing and has sufficient strength to absorb a downward pressure without damage to the implant.

### SUMMARY OF THE INVENTION

The present invention is an anatomic restoration dental Zirconium Dioxide implant having a milled shaft into which a titanium insert is press fit retained with members to prevent rotation of the titanium insert within the Zirconium Dioxide implant. The present invention is an improvement over the prior art by providing minimal contact between the stronger material such as titanium and the Zirconium Dioxide. Therefore, the present invention is an improvement over the prior art by substantially limiting the forces that are exerted on the portion of the implant made of Zirconium Dioxide. The insertion of the titanium insert allows the principal forces exerted on the two-piece Zirconium Dioxide implant to be received by the titanium insert portion and not the Zirconium Dioxide portion.

After the two-piece Zirconium Dioxide implant has been screwed into a patient's jawbone, a healing cap is affixed to the implant during a healing process. The healing cap has a beveled portion and a concave sidewall where the concavity is entirely around the sidewall. The concave portion encircles the healing cap and extends between the healing cap lower titanium member and the healing cap beveled portion and are innovative because the gum in this area will not recede and will become thicker as the tissue grows in because of the concave sidewall of the healing cap.

The present invention two-piece implant system includes a Zirconium Dioxide implant having a hollow milled chamber that receives a titanium insert. The titanium insert provides additional strength to the Zirconium Dioxide implant while still maintaining a predominantly Zirconium Dioxide (ZrO₂) composition. Zirconium Dioxide is commonly referred to as either Zirconium Oxide or Zirconium, however the proper chemical formula is ZrO₂ and correct name is Zirconium Dioxide.

The present invention includes a significant improvement in the healing cap which is placed onto the Zirconium Dioxide implant while the healing process takes place to enable the jaw bone to grow into the implant. Significant improvements in the healing cap include a concave exterior sidewall and a bevel top.

It is an object of the present invention to have a Zirconium Dioxide implant which has a milled shaft into which a titanium insert is press fit retained with members to prevent rotation of the titanium insert within the Zirconium Dioxide implant. The insertion of the titanium insert makes the implant a two a two-piece Zirconium Dioxide implant.

After the present invention two-piece Zirconium Dioxide implant has been screwed into a patient's jawbone, a healing cap is affixed to the implant during a healing time. The healing cap has a bevel portion and a sidewall with at least two oppositely disposed concave portions. Alternatively, the concave portions encircle the healing cap. With each variation, the concave portions extend between the healing cap lower titanium member and a healing cap beveled portion and are innovative because the gum in this area will not recede and will become thicker as the tissue grows in because of the concave sides of the healing cap. The beveled top of the healing cap enables tissue to grow around it during the healing process.

The titanium insert in the present invention also has a body that is tapered partially cylindrically shaped with a generally circular proximal end having two oppositely disposed female semicircular cutouts which respectively receive two oppositely disposed semicircular male protrusions of the Zirconium Dioxide base which extend into a central opening leading to a chamber into which the titanium insert is press fit retained. The two semicircular extensions of the Zirconium Dioxide base respectively mate with the two female semicircular cutouts in the titanium insert to prevent rotation when inserted into the open chamber within the Zirconium Dioxide implant.

The proximal end of the present invention two-piece implant system has the ability to receive a multitude of different attachments such as a titanium healing cap, impression coping and abutment. A distal end of each attachment has a non-round section. The proximal end of the titanium insert has a mating non-round interior portion to receive the non-round section of the attachment. Each mating non-round section/portion includes pentagonal shaped, hexagonal shaped and octagonal shaped. The mating non-round section/portion provide anti-rotation members to avoid the attachment rotating relative to the implant.

In addition, each added fixture such as a healing cap and abutment include a screw or bolt which extends through an opening in the healing cap/abutment, etc. and extends into the titanium insert. The screw or bolt has threads on at least a lower section which threads are threaded into mating threads on an interior sidewall of the titanium insert.

Further novel features and other objects of the present invention will become apparent from the following detailed description and discussion.

### BRIEF DESCRIPTION OF THE DRAWINGS

Referring particularly to the drawings for the purpose of illustration only and not limitation, there is illustrated:

FIG. 1 is a top/side perspective view in partial cross-section illustrating a Zirconium Dioxide implant with an interior cavity milled into it and a top with oppositely disposed male semicircular members protruding into an opening above the milled cavity;

FIG. 2 is a top/side perspective view of the present invention titanium insert having interior threads, an interior upper anti-rotation member which by way of example is an interior hexagonal shaped top section with an interior opening leading to the interior of the titanium insert, and female semicircular receiving members on its top circumferential wall configured to match the male semicircular protruding members in the Zirconium Dioxide implant illustrated in FIG. 1;

FIG. 3 is a cross sectional view taken when viewed from line 3-3 of FIG. 2, illustrating the internal threads in the titanium insert and the hexagonal anti-rotation member;

FIG. 4 is a top/side perspective view in partial cross-section illustrating the titanium insert press fit inserted into the milled cavity of the Zirconium Dioxide implant, with the interior threads on the interior circumferential wall of the titanium insert, also illustrating the upper interior hexagonal shaped top section and illustrating a respective female semicircular member of the titanium insert retained in a respective male semicircular protruding member of the Zirconium Dioxide implant;

FIG. 5 is a side perspective view in partial cross-section of a healing cap with a bottom section having an exterior hexagonal shape, a central shaft with exterior threads, a top bevel section, and a unique concave portion on the sidewall forming part of its circumferential wall;

FIG. 6 is a side perspective view in partial cross-section illustrating the healing cap affixed to the titanium insert of the Zirconium Dioxide implant, and the threaded screw of the healing cap partially threaded into the titanium insert within the Zirconium Dioxide implant;

FIG. 7 is a side perspective view of an impression coping with a bottom section having an anti-rotation portion which has an exterior hexagonal shape and a central shaft with exterior threads;

FIG. 8 is a side perspective view in partial cross-section, with the impression coping retained on the Zirconium Dioxide implant with the titanium insert and the screw from the impression coping partially threaded into the titanium insert;

FIG. 9 is a side perspective view of an abutment used with the present invention; and

FIG. 10 is a side perspective view of the abutment partially placed onto the Zirconium Dioxide implant.

### DETAILED DESCRIPTION OF THE PREFERRED EMBODIMENTS

Although specific embodiments of the present invention will now be described with reference to the drawings, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention.

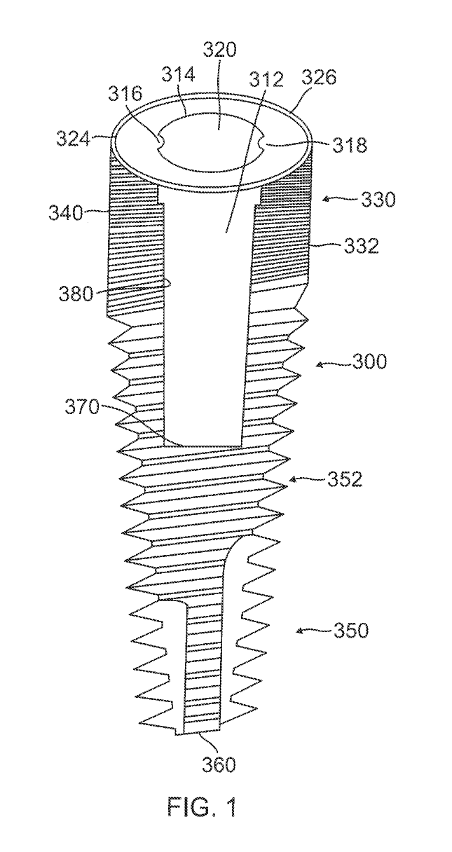

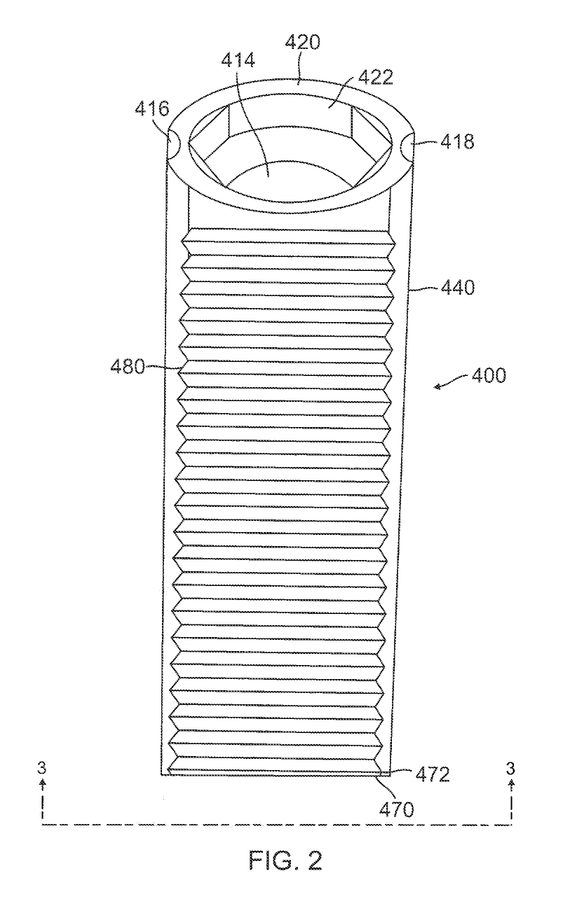

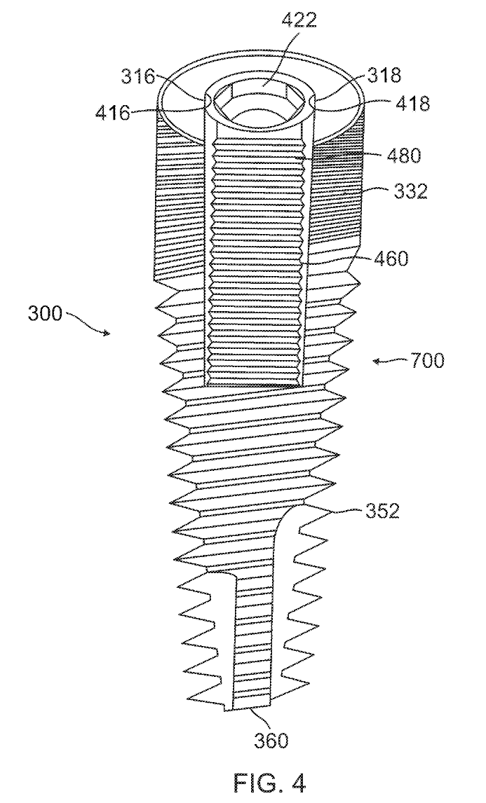

Referring to FIGS. 1 and 2, Zirconium Dioxide base piece 300 has an opening 320 leading to cavity 312 near the top. Zirconium Dioxide base piece 300 is made entirely of Zirconium Dioxide. Titanium insert 400 is press fit through opening 320 into cavity 312 of Zirconium Dioxide base piece 300 to form the present invention two-piece Zirconium Dioxide implant 700 (see FIG. 4).

Referring to FIG. 1, there is illustrated a top/side perspective view in partial cross-section illustrating a Zirconium Dioxide base piece 300 having a generally circular top surface 324 with a generally circular hollow chamber opening 320 in the center leading to open cavity or chamber 312 with interior sidewalls 380. Located on the circumferential edge of circular top surface 324 is a circumferential bevel 326. Circumferential bevel 326 is slightly higher than circular top surface 324 for the purpose of having a biological width. The purpose of having the circumferential bevel is to keep the micro movements at the junction of the implants and abutments away from the interior of the jawbone where the implant and jawbone meet so that the bone will be more stable and level relative to the implant. Circular top surface 324 contains oppositely disposed anti-rotation members, which by way of example, are parallel semi circular extensions or protrusions 316 and 318 that protrude into the top of generally circular chamber opening 320 which prevents rotation of a titanium insert 400 when the titanium insert 400 is inserted into a generally cylindrical hollow chamber 312 located within the Zirconium Dioxide base piece 300.

Further referring to FIG. 1, Zirconium Dioxide base piece 300 has a top cylindrical section 330 that surrounds a portion of cylindrical hollow chamber 312 and has top micro-threads 332 that extend from base outer circumferential wall 340 of top cylindrical section 330. Similarly, lower tapered section 350 has screw threads 352 that begin from the lowermost portion of the micro-threads 332 and continue to the distal end 360 of the Zirconium Dioxide base piece. 300. The micro-threads 332 and screw threads 352 create stability and grip to reduce the likelihood of the present invention two-piece Zirconium Dioxide implant 700 (see FIG. 4) from becoming dislodged when inserted into a patient's jawbone. Tapered section 350 of Zirconium Dioxide base piece 300 terminates at distal end 360. Similarly, cylindrical hollow chamber or cavity 312 terminates in hollow end wall 370.

Referring to FIG. 2, there is illustrated a top perspective view of titanium insert 400. Titanium insert 400 is made entirely of titanium or similar non brittle material with high strength. Titanium insert 400 has an internal chamber 414 and an insert distal end 470.

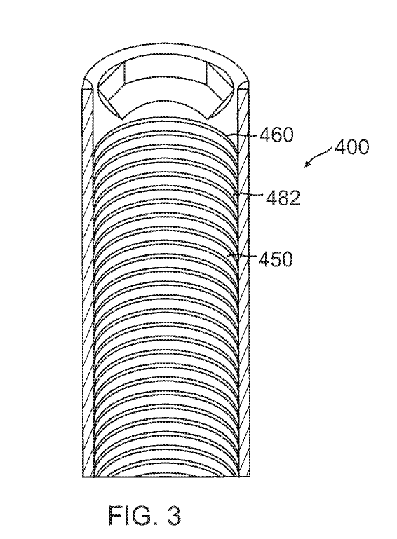

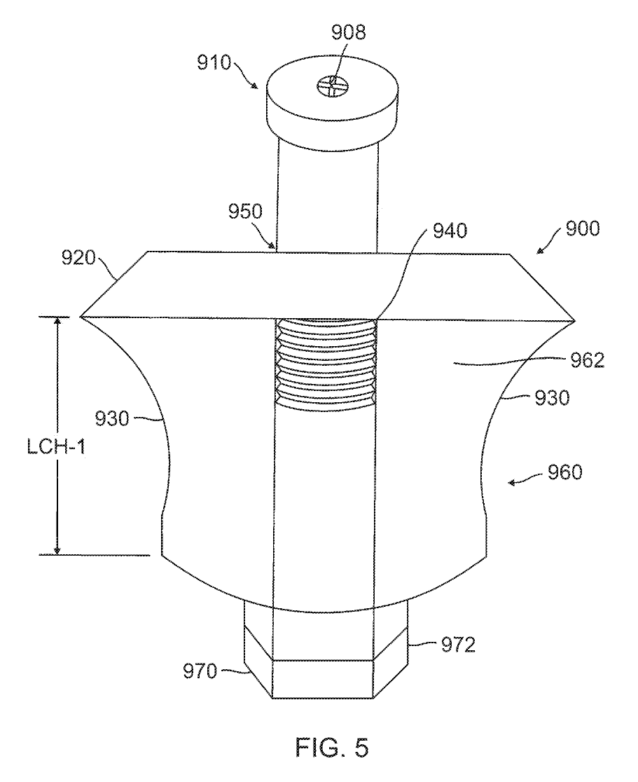

Referring to FIG. 3, there is illustrated a cross sectional view of the titanium insert when viewed from line 3-3 of FIG. 2. Insert internal threads 450 are on the entire interior circumferential wall 480 from the top 460 to the bottom 472. Insert internal threads 450 receive top screw 910 (illustrated in FIG. 5) to secure or affix one of the attachments disclosed in more detail herein.

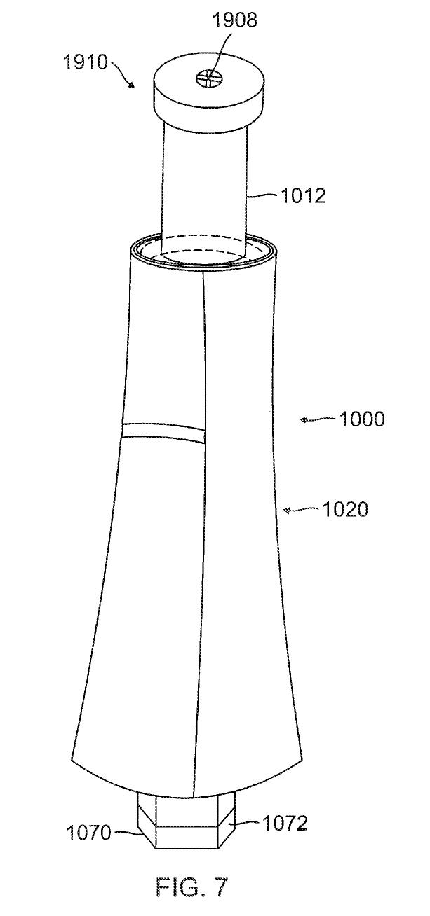

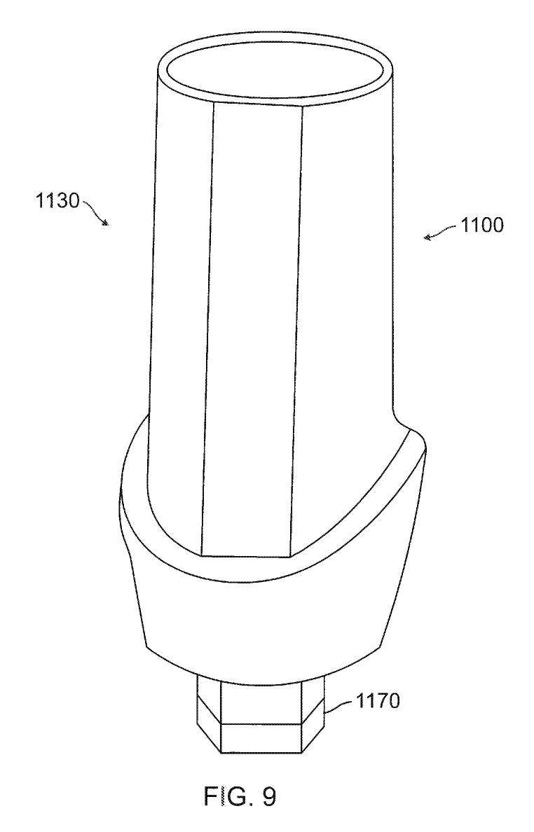

Referring to both FIGS. 2 and 3, the internal chamber 414 has an internal circumferential wall 480 so that when viewed in cross-section perspective as in FIG. 2, the exterior wall 480 appeared threaded but the threads 450 are on the interior 482 of the circumferential wall 480. The internal threads 450 within internal chamber 414 the that surround internal chamber 414 are for receiving a threaded screw from healing cap 900 (FIG. 5), a threaded screw from impression coping 1000 (FIG. 7), and a threaded screw from abutment 1100 (FIG. 9) when one is subsequently threaded into titanium insert 400. Above the top 460 of internal threads 450, the titanium inert 400 has an upper inner circumferential wall 422 that has a shape that corresponds to the shape of an abutment to be inserted.

By way of example, titanium insert 400 has an upper inner circumferential wall 422 in the shape of a hexagon that matches the hexagonal shape of a bottom of a healing cap 900 (FIG. 5), a bottom of an impression coping 1000 (FIG. 7), and a bottom of an abutment 1100 (FIG. 9) when one is subsequently inserted. The matching hexagonal shapes are anti-rotation shapes. It will be appreciated that hexagonal is 300 has a generally circular top surface 420 with parallel oppositely disposed anti-rotation members which by way of example are semicircular cutouts or recesses 416 and 418 which respectively receive protrusions 316 and 318 to prevent rotation when the titanium insert or plug 400 is inserted into milled interior chamber 312.

Titanium insert 400 has an upper inner circumferential wall 422 that has a shape that corresponds to the shape of an abutment to be inserted. By way of example, titanium insert 400 has an upper inner circumferential wall 422 in the shape of a hexagon that It will be appreciated that hexagonal is one anti-rotation shape and any non-round shape (square, pentagonal, octagonal, etc.) are within the spirit and scope of the present invention. Titanium insert 400 also has a lower inner circumferential wall 460 that contains insert internal threads 450 further defined in FIG. 3. In addition. Referring again to FIG. 2, titanium insert 400 has a smooth external sidewall 440.

Referring to FIG. 3, there is illustrated a cross sectional view of the matches the hexagonal shape of a bottom of a healing cap 900 (FIG. 5), a bottom of an impression coping 1000 (FIG. 7), and a bottom of an abutment 1100 (FIG. 9) when one is subsequently inserted. The matching hexagonal shapes are anti-rotation shapes. It will be appreciated that hexagonal is one anti-rotation shape and any non-round shape (square, pentagonal, octagonal, etc.) are within the spirit and scope of the present invention. Titanium insert 400 also has a lower inner circumferential wall 460 that contains insert internal threads 450 further defined in FIG. 3. In addition, Titanium insert 400 has a smooth external sidewall 440.

Referring to FIG. 3, there is illustrated a cross sectional view of the titanium insert when viewed from line 3-3 of FIG. 2. Insert internal threads 450 are on the entire interior circumferential wall 480 from the top 460 to the bottom 472. Insert internal threads 450 receive top screw 910 (illustrated in FIG. 5) to secure or affix one of the attachments disclosed in more detail herein.

Referring again to FIG. 2, the titanium insert 400 has a generally circular top surface 420 with parallel oppositely disposed anti-rotation members which by way of example are semicircular cutouts or recesses 416 and 418 which respectively receive protrusions 316 and 318 to prevent rotation when the titanium insert 400 is inserted into milled interior chamber 312. As stated above, titanium insert 400 hast an upper inner circumferential wall 422 that has a shape that corresponds to the shape of an abutment to be inserted. By way of example, titanium insert 400 has an upper inner circumferential wall 422 in the shape of a hexagon that It will be appreciated that hexagonal is one anti-rotation shape and any non-round shape (square, pentagonal, octagonal, etc.) are within the spirit and scope of the present invention. Titanium insert 400 also has a lower inner circumferential wall 460 that contains insert internal threads 450 further defined in FIG. 3. In addition. Referring again to FIG. 2, titanium insert 400 has a smooth external sidewall 440.

Referring to FIG. 4, there is illustrated a top/side perspective view in partial cross-section illustrating the titanium insert 400 press fit inserted into the milled cavity or chamber 312 of the Zirconium Dioxide base piece 300, with insert internal threads 480. Also illustrated is the upper interior hexagonal shaped top section 422, and a respective semicircular male protrusion 316 and 318 respectively retained in a respective semicircular female cutout 416 and 418 to prevent rotation of the titanium insert 400 in the chamber 312 of the Zirconium Dioxide implant base 300.

A further innovation of the present invention that is unique from the prior art, is the ability to have a screw from respective multiple inserts such as a healing cap, an impression coping, and an abutment that can be inserted and tightened with limited risk of damage to the Zirconium Dioxide base piece 300 during the tightening step.

In operation, a two-piece dental implant 700 comprising a Zirconium Dioxide base 300 and a titanium insert 400 is created by a mold wherein a milled opening 312 is made in the Zirconium Dioxide base piece 300 (also called Zirconium Dioxide implant base) which includes the components discussed in FIG. 1 and the titanium insert 400 (also called titanium insert plug) discussed in FIGS. 2 and 3 with the titanium insert 400 press fit retained into the cavity or chamber 312 and retained therein by mating anti-rotation members in the Zirconium Dioxide base and titanium insert as illustrated in FIG. 4. An opening is made in the patient's jawbone and the two-piece Zirconium Dioxide implant 700 is threaded into the patient's jawbone.

A key innovation of this invention is the insertion of the titanium insert 400 within the Zirconium Dioxide base piece 300 so that pressure can be placed upon the titanium portion of the present invention two-piece Zirconium Dioxide implant 700 while abn attachment is threaded into the titanium insert 400.

The healing process involves the jawbone growing in between micro-threads 332 and screw threads 352 of Zirconium Dioxide base piece 300 so that the implant is secure before a final crown is inserted.

Referring to FIG. 4, there is illustrated is a top/side perspective view in partial cross-section illustrating the titanium insert plug 400 press fit into the milled cavity 312 of the Zirconium Dioxide implant base piece 300 with the interior threads 450 on the interior circumferential wall 480 of the titanium insert 400. Also illustrated in FIG. 4 is the upper interior hexagonal shaped top section 422 of the titanium insert 400 and respective female semicircular members 416 and 418 of the titanium plug 400 retained in a respective male semicircular protruding member 316 and 318 of the two-piece reinforced Zirconium Dioxide implant 700. Together and collectively, these two-pieces, the zirconium Dioxide base piece 300 and the titanium insert 400 form the present invention two-piece Zirconium Dioxide implant 700 (illustrated together in FIG. 4).

The major improvement over the prior art in the present invention is to have a two-piece system that includes a titanium insert 400 (illustrated in FIG. 2) that is placed within Zirconium Dioxide base piece 300 (illustrated in FIG. 1) to form the present invention two-piece Zirconium Dioxide implant 700 (illustrated in FIG. 4). The key difference between the present invention and the prior art is (1) the tightening of the screw from each respective one of the multiple removably affixed attachments used with the present invention are retained metal screw of attachment to metal of insert, (2) the retention is threaded metal to metal and no metal threads are affixed to the Zirconium Dioxide based 300, and (3) if there is inadvertent bite down pressure during the healing time or crown preparation time, the pressure is absorbed by the titanium insert to reduce the possible cracking of the Zirconium Dioxide base.

Referring to FIG. 5, there is illustrated is a side perspective view in partial cross-section of a healing cap 900 with a titanium healing cap bottom section 970 having an anti-rotational shape which by way of example has a hexagonal sidewall 972 configured to be inserted into the hexagonal sidewall 422 of the titanium insert plug 400. A top screw or bolt 910 having exterior screw threads 940 is used to affix healing cap 940 to titanium insert 400 with the threads 940 of threaded bolt 910 threaded into the threads 450 of the titanium insert plug 400. Healing cap 900 also includes a top beveled section 920 and a unique concave portion 930 forming part of its circumferential wall 960 of the body 962 of healing cap 900. The circumferential bevel 930 entirely encircles the healing cap body 962 and extends between the titanium healing cap bottom section 970 and the healing cap beveled portion 920 and are innovative because the gum in this area will not recede and will become thicker as the tissue grows in because of the concave sides of the healing cap. Top beveled section 920 of the healing cap enables tissue to grow around it during the healing process. Therefore, a second significant innovation of the present invention is having concave sidewalls, either partially or entirely around the healing cap body 962.

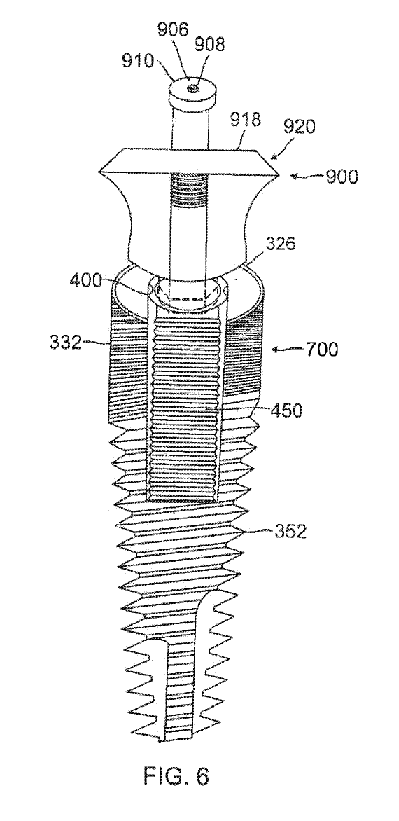

Referring to FIG. 6, there is illustrated a side perspective view in partial cross-section illustrating the healing cap 900 partially threaded into the titanium insert 400 of the present invention two-piece Zirconium Dioxide implant 700. The top screw of bolt 910 has a female portion 908 to receive the head of a screwdriver (Phillips® or flat).

Further referring to FIGS. 5 and 6, the next process is to place a titanium healing cap 900 into titanium insert 400 to enable the tissue of the jawbone to grow into bottom teeth or ridges 352 of the present invention two-piece Zirconium Dioxide implant 700. The healing cap 900 (FIG. 5) has a central opening 950 that allows top screw 910 to fit through central opening 950 and affix titanium healing cap 900 to titanium insert 400 by means of top screw external threads 940 mating with insert internal threads 450. The length of top screw threads 940 are shown by way of example as illustrated in FIGS. 5 and 6. These threads can cover the entire shaft length or a portion thereof. Also illustrated in FIG. 5 is a healing cap beveled portion 920, two concaved portions 930, and a healing cap lower titanium member 970. The two concave portions 930 that encircle tat least a portion of the healing cap 900 and extend between healing cap lower titanium member 970 and a healing cap beveled portion 920 are innovative because the gum in this area will not recede and will become thicker as the tissue grows in because of the concave shape.

In summary, the Zirconium Dioxide base piece 300 with titanium insert 400 is threaded into the patient's jawbone and thereafter the top screw 910 of the healing cap 900 is threaded into the threads 450 of the titanium insert 400 and the top 906 of top screw 910 is flush with the top 918 of the bevel 920. On top of the bevel of a healing cap a fake tooth or crown is placed so that it is either bonded to adjacent teeth or a removable appliance. It is important that no direct pressure is placed onto the healing cap. In addition, the concave portion of the healing cap (which can be made of either titanium or Zirconium Dioxide) is unique because it allows the gum to grow into it so when the crown is placed in, the gum will not recede. The bevel 920 is titanium and has roughly a 45 degree angle so that tissue can grow around it because the bevel is at the gum line and there are no sharp corners. Then, the fake tooth or crown is put over the healing cap and affixed as described so that no pressure is put on the two-piece Zirconium Implant 700 while the jawbone and gum tissue is healing.

Further referring to FIGS. 5 and 6, there is illustrated the healing cap 900 positioned above and being affixed to the two-piece Zirconium Dioxide implant 700 by means of top screw 910. After a period of time, such as 4 or 5 months when the bone and tissue have grown into bottom teeth or ridges 352 and top ridges 332 of two-piece Zirconium Dioxide implant 700, titanium healing cap 900 is removed and replaced with a titanium impression coping 1000 (illustrated in FIG. 7) which is then placed into and onto the two-piece Zirconium Dioxide implant 700 in place of titanium healing cap 900.

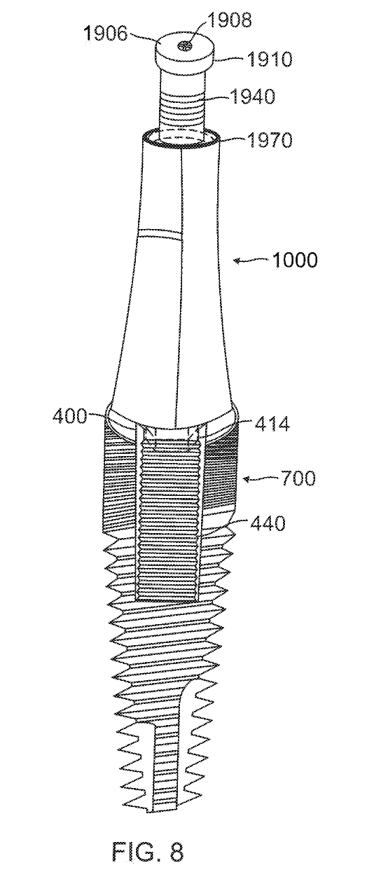

Referring to FIG. 7, there is illustrated a side perspective view of an impression coping 1000 with an impression coping bottom section 1070 made of titanium with a hexagonal shape exterior surface which is received within hexagonal surface 422 of titanium insert plug 400. The impression coping 1000 has a sidewall 1020. Referring to FIG. 8, after impression coping bottom section 1070 is inserted, top threaded screw 1910 with exterior threads comparable to threads 940 is used to affix impression coping 1000 to titanium insert 400. Top screw 1910 has a top surface 1908 with an indent 1906 to receive a screwdriver head (Phillips® or flat).

Referring to FIG. 8, there is illustrated a side perspective view in partial cross-section, with the impression coping 1000 partially inserted and top screw 1910 partially threaded into threads 450 of titanium insert plug 400 of the two-piece Zirconium Dioxide implant 700.

Referring to FIGS. 7 and 8, titanium impression coping 1000 has a coping lower titanium member 1070 with anti-rotation surface 1072 that fits within anti-rotation surface 422 of titanium insert 400. The length of top screw threads 1940 on top screw 1910 enables top surface 1908 to be flush with top surface 1970 of impression coping 1000.

In operation, after about 5 months the healing process is completed, the healing cap is removed and replaced with the impression coping inserted as discussed above. The dentist then has impression material in a tooth tray and the person bites down onto and into the impression material with the impression coping in place. The impression is used to make the crown. The impression coping is removed and placed into the impression material and the impression in the impression material and impression coping 1000 are sent to a laboratory to have the crown made and the healing cap is put back onto the two-piece Zirconium Dioxide implant 700 while the crown and abutment are being made in a laboratory.

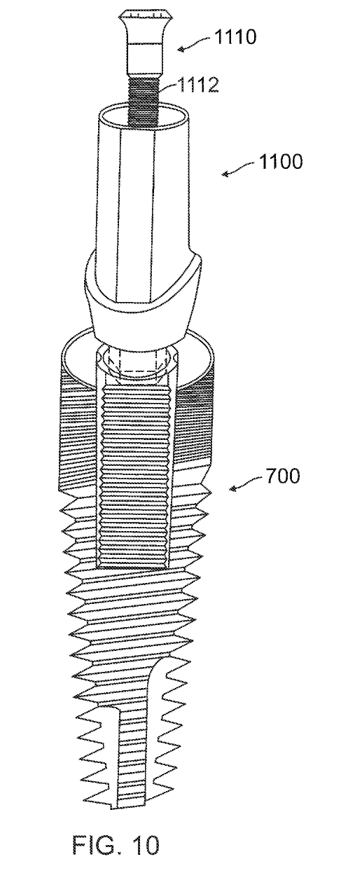

Referring to FIG. 9, there is illustrated a side perspective view of an abutment 1100. Referring to FIG. 10, there is illustrated a side perspective view of the abutment 1100 partially placed onto the Zirconium Dioxide implant 700.

Referring to FIGS. 9 and 10, a crown is then made from titanium impression coping 1000. After the crown has been completed, the titanium impression coping 1000 is removed and replaced with an abutment 1100 (FIG. 9) which has the same shaped titanium abutment bottom section 1170 that matches insert circumferential wall 422 (FIG. 2) of titanium insert 400. The abutment upper portion 1130 is made of Zirconium Dioxide and forms the base or post where the final crown is mounted. The abutment 1100 is retained by a titanium retaining screw 1110 with threads 1112 which is inserted into and threaded through the abutment 1100 and threaded into and onto threads 450 of titanium insert plug 400 to affix the abutment 1100 to the two-piece Zirconium Dioxide implant 700. Thereafter, the result is the abutment with the crown that matches.

A key innovation is that metal screws and metal healing cap and metal impression coping only come in contact with the metal insert plug 400 which is preferably made of titanium. The metal is concealed by the attractive, yet brittle, Zirconium Dioxide.

The circumference shapes used in the connection of inner circumferential wall 422 and corresponding hexagonal ends are byway of example only and it is within the spirit and scope of this invention for these shapes to be a multitude of different shapes that would perform the same anti-rotation function including but not limited to triangle, rectangle, pentagonal, hexagonal octagonal, rhombus, or trapezoid.

It is further within the spirit and scope of this invention to have other material used for the two-piece system. The Zirconium Dioxide base could be made of similar ceramic materials or similar materials that are suitable for placing in the jawbone for purposes of the fixture. Similarly, the titanum insert and corresponding titanium ends of the attachments described above can be made of materials other than titanium that have similar superior strength characteristics. The abutment 1100 can be made of metal such as titanium or can be made of Zirconium Dioxide.

Of course the present invention is not intended to be restricted to any particular form or arrangement, or any specific embodiment, or any specific use, disclosed herein, since the same may be modified in various particulars or relations without departing from the spirit or scope of the claimed invention herein above shown and described of which the apparatus or method shown is intended only for illustration and disclosure of an operative embodiment and not to show all of the various forms or modifications in which this invention might be embodied or operated.

1-20. (canceled)

21-31. (canceled)

32. A two-piece dental implant including:
a. a zirconium dioxide base piece made of zirconium dioxide having a generally circular top surface with a circumferential bevel located on a circumferential edge of the generally circular top surface, the circumferential bevel higher than the generally circular top surface;
b. a generally circular hollow chamber opening in the center of the generally circular top surface, the generally circular hollow chamber opening leading to a generally cylindrical hollow chamber located within the zirconium dioxide base piece, the generally cylindrical hollow chamber has interior sidewalls and terminates in a flat end wall, the generally circular top surface including oppositely disposed anti-rotation members protruding into the generally circular hollow chamber opening;
c. a titanium insert made of titanium having an internal chamber with an interior circumferential wall with internal threads on the interior circumferential wall from a top to a bottom, the titanium insert having a smooth exterior sidewall of the interior circumferential wall having a flat distal end, the titanium insert is press fit inserted into and retained in the generally cylindrical hollow chamber of the zirconium dioxide base piece, the titanium insert has a generally circumferential top surface with parallel oppositely disposed anti-rotation members which respectively receive an anti-rotation member of the generally circular top surface of the zirconium diode base piece, the flat distal end of the titanium insert received and accepted by the flat end wall of the cylindrical hollow chamber of the zirconium dioxide base piece, the titanium insert including a non-round upper inner circumferential wall within the generally circular top surface; and
d. the zirconium diode base piece has a top cylindrical section that surrounds a portion of the generally cylindrical hollow chamber and includes top micro-threads that extend from a base outer circumferential wall through top cylindrical section, a lower tapered section of the zirconium dioxide base piece includes exterior screw threads that begin from a lowermost portion of said micro-threads and continue to an exterior distal end of the zirconium dioxide base piece;
e. wherein, a titanium screw is used in conjunction with the two-piece dental implant which is adapted to be implanted into a jawbone bone configured to non-rotatably receive in succession a healing cap, an impression coping and an abutment, each of which is retained in titanium insert by a titanium screw having exterior threads which match the threads on the internal circumferential wall surface of the titanium insert, and the titanium screw does not touch any portion of the zirconium dioxide base piece.

33. The two-piece dental implant in accordance with claim 32, further comprising:
a. a healing cap including a top bevel section extending into a body having a circumferential concave sidewall entirely encircling the healing cap, the body extending to a bottom section having a non-round shape in an exterior sidewall respectively retained within said non-round shape sidewall which conforms to and is received with the non-round upper inner circumferential wall within the generally circumferential top surface of the titanium insert; and
b. said titanium screw extending through a channel in a longitudinal center of the healing cap extending into the internal chamber of the titanium insert, the exterior threads of the titanium screw threaded into the threads of the internal circumferential wall of the titanium insert.

34. The two-piece dental implant in accordance with claim 32, further comprising:
a. an impression coping with a body having a top surface extending to a sidewall extending to an impression coping bottom section made of titanium with a bottom section having a non-round shape in an exterior sidewall respectively retained within said non-round shape sidewall which conforms to and is received with the non-round upper inner circumferential wall within the generally circumferential top surface of the titanium insert; and
b. said titanium screw extending through a channel in a longitudinal center of the impression coping extending into the internal chamber of the titanium insert, the exterior threads of the titanium screw threaded into the threads of the internal circumferential wall of the titanium insert.

35. The two-piece dental implant in accordance with claim 32, further comprising:
a. an abutment made of zirconium dioxide including a bottom section made of titanium with a bottom section having a non-round shape in an exterior sidewall respectively retained within said non-round shape sidewall which conforms to and is received with the non-round upper inner circumferential wall within the generally circumferential top surface of the titanium insert; and
b. said titanium screw extending through a channel in a longitudinal center of the abutment extending into the internal chamber of the titanium insert, the exterior threads of the titanium screw threaded into the threads of the internal circumferential wall of the titanium insert.

36. The two-piece dental implant in accordance with claim 32, further comprising:
a. the anti-rotation members of the titanium insert are female semi-circular cutouts; and
b. the anti-rotation members of the zirconium dioxide base piece are semi-circular male protrusions.

37. The two-piece dental implant in accordance with claim 32, further comprising: the non-round upper inner circumferential wall within generally circumferential top surface is hexagonal in shape.

38. A two-piece dental implant including:
a. a zirconium dioxide base piece made of zirconium dioxide having a generally circular top surface with a circumferential bevel located on a circumferential edge of the generally circular top surface, the circumferential bevel higher than the generally circular top surface;
b. a generally circular hollow chamber opening in the center of the generally circular top surface, the generally circular hollow chamber opening leading to a generally cylindrical hollow chamber located within the zirconium dioxide base piece, the generally cylindrical hollow chamber has interior sidewalls and terminates in a flat end wall, the generally circular top surface including oppositely disposed anti-rotation members protruding into the generally circular hollow chamber opening;
c. a titanium insert made of titanium having an internal chamber with an interior circumferential wall with internal threads on the interior of the circumferential wall from a top to a bottom, the titanium insert having a smooth exterior sidewall of the interior circumferential wall having a flat distal end, the titanium insert is press fit inserted into and retained in the generally cylindrical hollow chamber of the zirconium dioxide base piece, the titanium insert has a generally circumferential top surface with parallel oppositely disposed anti-rotation members which respectively receive an anti-rotation member of the generally circular top surface of the zirconium diode base piece, the flat distal end of the titanium insert received and accepted by the flat end wall of the cylindrical hollow chamber of the zirconium dioxide base piece, the titanium insert including a non-round upper inner circumferential wall within the generally circular top surface; and
d. the zirconium diode base piece has a top cylindrical section that surround a portion of the generally cylindrical hollow chamber and includes top micro-threads that extend from a base outer circumferential wall through top cylindrical section, a lower tapered section of the zirconium dioxide base piece includes exterior screw threads that begin from a lowermost portion of said micro-threads and continue to an exterior distal end of the zirconium dioxide base piece.

39. A two-piece dental implant including:
a. a zirconium dioxide base piece made of zirconium dioxide having a generally circular top surface with a circumferential bevel located on a circumferential edge of the generally circular top surface, the circumferential bevel higher than the generally circular top surface;
b. a generally circular hollow chamber opening in the center of the generally circular top surface, the generally circular hollow chamber opening leading to a generally cylindrical hollow chamber located within the zirconium dioxide base piece, the generally cylindrical hollow chamber has interior sidewalls and terminates in a flat end wall, the generally circular top surface including oppositely disposed anti-rotation members protruding into the generally circular hollow chamber opening; and
c. a titanium insert made of titanium having an internal chamber with an interior circumferential wall with internal threads on the interior of the circumferential wall from a top to a bottom, the titanium insert having a smooth exterior sidewall of the interior circumferential wall having a flat distal end, the titanium insert is press fit inserted into and retained in the generally cylindrical hollow chamber of the zirconium dioxide base piece, the titanium insert has a generally circumferential top surface with parallel oppositely disposed anti-rotation members which respectively receive an anti-rotation member of the generally circular top surface of the zirconium diode base piece, the flat distal end of the titanium insert received and accepted by the flat end wall of the cylindrical hollow chamber of the zirconium dioxide base piece, the titanium insert including a non-round upper inner circumferential wall within the generally circular top surface.

40. The two-piece dental implant in accordance with claim 39, further comprising: the zirconium diode base piece has a top cylindrical section that surround a portion of the generally cylindrical hollow chamber and includes top micro-threads that extend from a base outer circumferential wall through top cylindrical section, a lower tapered section of the zirconium dioxide base piece includes exterior screw threads that begin from a lowermost portion of said micro-threads and continue to an exterior distal end of the zirconium dioxide base piece.
